(19) United States
(12) Patent Application Publication
(10) Pub. No.: US 2025/0315800 A1
(43) Pub. Date: Oct. 9, 2025

(54) INTELLIGENT DATA PLATFORM FOR MEDICAL WASTE MANAGEMENT AND EQUIPMENT MONITORING

(71) Applicant: ZHEJIANG WEIDUN ENVIRONMENTAL TECHNOLOGY CO., LTD., Hangzhou (CN)

(72) Inventors: Weixing CHEN, Hangzhou (CN); Yiming CHEN, Hangzhou (CN); Guanzhong HUANG, Hangzhou (CN)

(73) Assignee: ZHEJIANG WEIDUN ENVIRONMENTAL TECHNOLOGY CO., LTD., Hangzhou (CN)

(21) Appl. No.: 19/172,154

(22) Filed: Apr. 7, 2025

(30) Foreign Application Priority Data
Apr. 7, 2024 (CN) 202410406960.1

(51) Int. Cl. G06Q 10/30; G06N 3/042; G06N 3/08; G16H 40/40

(52) U.S. Cl. CPC G06Q 10/30; G06N 3/042; G06N 3/08; G16H 40/40

(57) ABSTRACT

An intelligent data platform for medical waste management and equipment monitoring includes: a data collection module configured to collect medical waste information and equipment processing data; a data processing module configured to train and process the collected medical waste information and equipment processing data; a data storage module configured to store the trained and processed datasets; a data feedback and monitoring module configured to provide feedback and monitoring based on the trained and processed datasets; and a human-machine interaction terminal configured to display data and receive user input commands.

### CROSS-REFERENCE TO RELATED APPLICATIONS

The present application claims priority to Chinese Patent Application No. 202410406960.1, filed on Apr. 7, 2024, the content of which is incorporated herein by reference in its entirety.

### TECHNICAL FIELD

The present application relates to the technical field of medical waste management, specifically to an intelligent data platform for medical waste management and equipment monitoring.

### BACKGROUND

In the medical field, with the increasing frequency of medical activities, the generation of medical waste has grown year by year. Due to its hazardous nature, improper handling of medical waste may pose significant threats to the environment and human health. Therefore, effective management and safe disposal of medical waste are critically important.

However, traditional methods for statistical data collection and processing of medical waste predominantly rely on manual operations. Staff spend considerable time manually recording and editing key information such as the type, quantity, and source of medical waste, resulting in inefficiency and susceptibility to errors. Additionally, manually recorded data storage methods suffer from limitations, including vulnerability to data loss, difficulties in long-term preservation, and challenges in rapid retrieval, which further increase the complexity and risks of data management.

More critically, manual statistical approaches fail to accurately trace the source of medical waste generation. Once issues arise, it is difficult to promptly identify the origin and implement corrective measures. Furthermore, delayed information feedback hinders the timeliness and effectiveness of medical waste disposal.

For medical waste treatment equipment, effective monitoring is also challenging, which increases operational risks. For instance, untimely replacement of components may lead to ineffective or delayed waste treatment, resulting in significant accumulation of medical waste.

In view of the above, there is a need in the art for an intelligent data platform for medical waste management and equipment monitoring to address these issues.

### SUMMARY

To resolve the technical problems in the prior art, namely, the inefficiency of manual data collection for medical waste and inadequate monitoring of medical waste treatment equipment that adversely impacts operational efficacy, the present application provides an intelligent data platform for medical waste management and equipment monitoring.

The platform comprises:
a data collection module configured to collect medical waste information and equipment processing data;
a data processing module configured to train and process the collected medical waste information and equipment processing data;
a data storage module configured to store the trained and processed datasets;
a data feedback and monitoring module configured to provide feedback and monitoring based on the trained and processed datasets; and
a human-machine interaction terminal configured to display data and receive user input commands.

Preferably, the data processing module comprises a convolutional neural network (CNN) module and a graph convolutional neural network (GCN) module, wherein:
the CNN module is configured to train and process the medical waste information;
and the GCN module is configured to train and process the equipment processing data.

Preferably, the medical waste information includes one or more of the following: category information, regional information, hospital information, department information, medical waste type information, department-specific collection frequency, and total quantities of various medical wastes.

Preferably, the steps for training and processing medical waste information by the CNN module include:
Performing data preprocessing on various types of medical waste information;
Constructing a CNN model, wherein the CNN model comprises a convolutional layer, a pooling layer, and a fully connected layer;
Inputting preprocessed data into the CNN model and performing forward propagation calculations through the convolutional layer, pooling layer, and fully connected layer to obtain output data;
Calculating the difference between the model's output data and actual values using a predefined loss function;
Updating the weights and biases of the CNN model using an optimization algorithm based on the loss function gradient;
Iteratively optimizing the CNN model until a first iteration condition is met; and
Outputting the trained CNN model.

Preferably, the first iteration condition is convergence of the CNN model or reaching a preset number of iterations.

For the output of the convolutional layer:

$${O}_{i}^{( l )} = {\sum }_{j = 0}^{k - 1} {l}_{i + j}^{( l - 1 )} \cdot {K}_{j}^{( l )} \qquad ( 1 )$$

wherein Oᵢ^{(l)} is the output at position i of the l-th convolutional layer, Iᵢ₊ⱼ^{(L}^{₋₁}⁾ is the input at position i+j of the (l−1)-th layer, Kⱼ^{(l)} is the weight of the l-th layer's convolutional kernel at offset j, and k is the size of the convolutional kernel.

For the output of the activation function:

$${A}_{i}^{( l )} = f ( {O}_{i}^{( l )} ) \qquad ( 2 )$$

wherein f is the activation function, and Aᵢ^{(l)} is the activated output at position i of the l-th layer.

For the output of the pooling layer:

$${P}_{i}^{( l )} = {max}_{j = 0}^{s - 1} {A}_{i \times s + j}^{( l )} \qquad ( 3 )$$

wherein Pᵢ^{(l)} is the output at position i of the l-th pooling layer, s is the stride of the pooling operation, and Equation (3) represents selecting the maximum value within each region of size S.

For the output of the fully connected layer:

$$y = W \cdot x + b \qquad ( 4 )$$

wherein y is the output of the fully connected layer, W is the weight matrix, x is the input feature vector, and b is the bias term.

Preferably, the equipment processing data includes one or more of the following: daily operation count of equipment, single-operation duration, pre-operation medical waste weight, pre-operation medical waste volume, post-operation medical waste weight, and post-operation medical waste volume.

Preferably, the steps for training and processing equipment processing data by the GCN module include:
Preprocessing each data item in the equipment processing data, wherein each device is represented as a node, and the feature vector of each device includes its respective data;
Constructing a GCN model;
Inputting preprocessed data into the GCN model and propagating/updating node features layer-by-layer via graph convolution until final node representations are obtained;
Calculating the difference between the model's output data and actual values using a predefined loss function;
Updating the weight parameters of the GCN model based on the loss function gradient;
Iteratively optimizing the GCN model until a second iteration condition is met; and Outputting the trained GCN model.

Preferably, the second iteration condition is convergence of the GCN model or reaching a preset number of iterations.

Preferably, the formula for the GCN model includes:

$${H}^{( l + 1 )} = σ ( Â {H}^{( I )} {W}^{( I )} ) \qquad ( 5 )$$

$$Â = {D}^{\frac{1}{2}} ( A + I ) {D}^{\frac{1}{2}} \qquad ( 6 )$$

wherein H^{(l)} is the node feature matrix of the l-th layer (each row corresponds to a node's feature vector), A is the normalized version of the graph's adjacency matrix A, D is the degree matrix, I is the identity matrix, W^{(h)} is the learnable weight matrix of the l-th layer, and σ is the activation function.

The intelligent data platform for medical waste management and equipment monitoring provided by the present application achieves comprehensive and efficient management of medical waste information and equipment processing data by integrating multiple functional modules, including data collection, processing, storage, feedback, and monitoring.

As demonstrated above, the intelligent data platform of the present application offers the following technical advantages:

Enhanced management efficiency: Automated data collection and intelligent processing significantly reduce manual intervention, improving operational efficiency.

Improved data accuracy: Utilization of algorithms and models ensures higher data accuracy and reliability.

Data security assurance: Reliable storage technologies and security measures safeguard data integrity and safety.

Real-time monitoring and feedback: The data feedback and monitoring module enables real-time oversight of waste processing and equipment operation, allowing prompt managerial responses.

Superior user experience: The human-machine interaction terminal provides intuitive data visualization, enabling users to easily track processing status and operational metrics.

### BRIEF DESCRIPTION OF THE DRAWINGS

The features and advantages of the present application will become clearer through the accompanying drawings, which are illustrative and not to be construed as limiting. In the drawings:

FIG. 1 is a schematic structural diagram of the intelligent data platform for medical waste management and equipment monitoring according to the present application.

FIG. 2 is a schematic structural diagram of the data processing module of the intelligent data platform according to the present application.

### DETAILED DESCRIPTION OF THE EMBODIMENTS

To clarify the objectives, technical solutions, and advantages of the embodiments of the present application, the technical solutions in the embodiments of the application will be described clearly and comprehensively below in conjunction with the accompanying drawings. It is evident that the described embodiments represent a subset of the application's embodiments rather than an exhaustive list. All other embodiments derived by those skilled in the art based on the present application without creative effort shall fall within the scope of protection of the application.

Addressing the issues identified in the Background Art-specifically, the inefficiency of manual data collection for medical waste and inadequate monitoring of medical waste treatment equipment leading to operational risks—the present application provides an intelligent data platform for medical waste management and equipment monitoring. This platform aims to enhance medical waste management efficiency, ensure data security, enable real-time monitoring and feedback, and meet diverse management requirements.

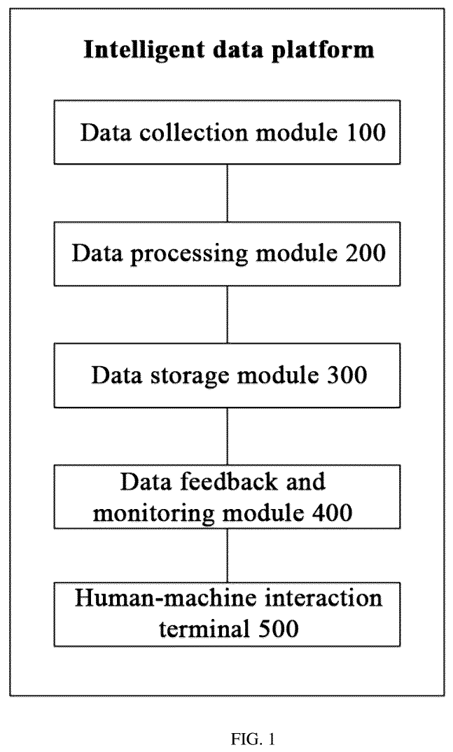

As shown in FIG. 1, the intelligent data platform of the application comprises:
a data collection module 100 configured to collect medical waste information and equipment processing data;
a data processing module 200 configured to train and process the collected medical waste information and equipment processing data;
a data storage module 300 configured to store the trained and processed datasets;
a data feedback and monitoring module 400 configured to provide feedback and monitoring based on the trained and processed datasets; and
a human-machine interaction terminal 500 configured to display data and receive user input commands.

In the above configuration, the platform first automates real-time collection of medical waste information and equipment processing data via the data collection module 100, eliminating the inefficiencies and errors inherent in traditional manual methods, thereby significantly improving data accuracy and collection efficiency. Second, the data processing module 200 intelligently trains and processes the collected data to extract valuable insights and features, providing robust support for subsequent decision-making and analysis. Continuous optimization of training models further enhances the platform's processing capabilities. Third, the data storage module 300 employs advanced storage technology to ensure secure, long-term preservation of datasets while enabling rapid retrieval, effectively resolving issues such as data loss and poor manageability associated with manual recording. Fourth, the data feedback and monitoring module 400 delivers real-time feedback and monitoring based on processed datasets, enabling managers to promptly track waste treatment progress and equipment operational status. Upon detecting anomalies, the platform triggers immediate responses to ensure safe waste disposal and stable equipment operation. Finally, the human-machine interaction terminal 500 offers intuitive data visualization through charts and dashboards, allowing users to monitor waste treatment and equipment metrics effortlessly. Users may also input commands via the terminal to flexibly operate and control the platform, meeting diverse management needs.

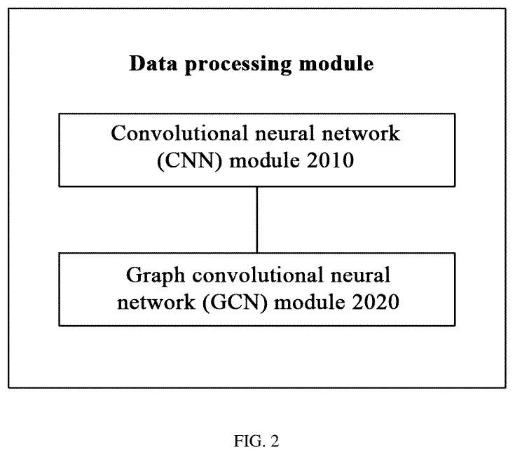

Preferably, as illustrated in FIG. 2, the data processing module 200 comprises a convolutional neural network (CNN) module 2010 and a graph convolutional neural network (GCN) module 2020.

The CNN module 2010 is configured to train and process medical waste information. Leveraging CNN's robust feature extraction and classification capabilities, this module accurately identifies and categorizes critical medical waste attributes such as type, quantity, and source. In one implementation, the medical waste information includes one or more of the following: category information, regional information, hospital information, department information, medical waste type, department-specific collection frequency, and total waste quantities. For example:

Department information may be acquired by scanning QR codes on department signage.

Medical waste type information (e.g., pathological, sharps, infectious waste) may be obtained by scanning QR codes on waste bags.

This setup enables precise traceability of medical waste, enhancing data processing accuracy. The human-machine interaction terminal 500 may display alerts for regional or hospital-specific anomalies (e.g., abnormal trends in infectious, sharps, or pathological waste) and supports manual oversight.

The GCN module 2020 is configured to train and process equipment processing data. GCNs are particularly suited for analyzing data with complex topological relationships. When processing equipment data, this module effectively captures interdependencies among devices to predict operational status, fault warnings, and processing efficiency. In one implementation, the equipment processing data includes one or more of the following: daily operation count, single-operation duration, pre-operation medical waste weight/volume, and post-operation medical waste weight/volume. The human-machine interaction terminal 500 allows users to adjust critical equipment parameters, display optimization recommendations, and issue alerts for component replacements (e.g., parts requiring replacement after a specified number of operations).

In some preferred embodiments, the steps for training and processing medical waste information include:
S10: Perform data preprocessing on various types of medical waste information. Specifically, data preprocessing further comprises:
S101: Data cleansing-Remove duplicate, invalid, or anomalous data to ensure accuracy and consistency.
S102: Data transformation-Convert raw data into formats suitable for neural network processing (e.g., transforming images into pixel matrices or text into word vectors).
S103: Data normalization/standardization-Adjust data ranges to a uniform scale to eliminate dimensional disparities between features and accelerate model convergence.

In optional embodiments, a data augmentation step may follow S103:
S104: Data augmentation-Increase data diversity via rotation, flipping, or scaling to enhance model generalization.
S11: Construct a convolutional neural network (CNN) model, wherein the CNN comprises convolutional layers, pooling layers, and fully connected layers.

For the convolutional layer output:

$${O}_{i}^{( l )} = {\sum }_{j = 0}^{k - 1} {l}_{i + j}^{( l - 1 )} \cdot {K}_{j}^{( λ )} \qquad ( 1 )$$

wherein Oᵢ^{(l)} is the output at position i of the l-th convolutional layer, Iᵢ₊ⱼ^{(L}^{₁}⁾ is the input at position i+j of the (l−1)-th layer, Kⱼ⁽¹⁾ is the weight of the l-th layer's convolutional kernel at offset j, and k is the kernel size.

For the activation function output:

$${A}_{i}^{( l )} = f ( {O}_{i}^{( l )} ) \qquad ( 2 )$$

wherein f is the activation function, and Aᵢ^{(l)} the activated output at position i of the l-th layer.

For the pooling layer output:

$${P}_{i}^{( l )} = {max}_{j = 0}^{s - 1} {A}_{i \times s + j}^{( l )} \qquad ( 3 )$$

wherein Pᵢ^{(l)} is the output at position i of the l-th pooling layer, and s is the pooling stride. Equation (3) selects the maximum value within each s-sized region.

For the fully connected layer output:

$$y = W \cdot x + b \qquad ( 4 )$$

wherein y is the output, W is the weight matrix, x is the input feature vector, and b is the bias term.
S12: Input preprocessed data into the CNN model and perform forward propagation through the convolutional, pooling, and fully connected layers to generate output data.
S13: Calculate the discrepancy between the model's output and actual values using a predefined loss function (e.g., cross-entropy loss, mean squared error) to quantify prediction errors.
S14: Update the CNN model's weights and biases via optimization algorithms (e.g., gradient descent, Adam) based on the loss function gradient to minimize prediction errors.
S15: Iteratively optimize the CNN model until a first iteration condition is met. Preferably, the first iteration condition is model convergence or reaching a preset iteration count.
S16: Output the trained CNN model.

In some preferred embodiments, the steps for training and processing equipment processing data include:
S20: Preprocess equipment processing data, wherein each device is represented as anode, and each node's feature vector comprises the device's data.

Specifically, preprocessing further comprises:
S201: Feature extraction-Extract device-specific data such as daily operation count, single-operation duration, pre-/post-operation waste weight/volume.
S202: Remove anomalies, missing values, and duplicates to ensure data accuracy.
S203: Construct a device graph based on inter-device connectivity or similarity, wherein nodes represent devices and edges represent relationships.
S204: Normalize feature vectors to eliminate dimensional disparities.
S21: Construct a graph convolutional neural network (GCN) model.

The GCN formulas include:

$${H}^{( l + 1 )} = σ ( Â {H}^{( h )} {W}^{( l )} ) \qquad ( 5 )$$

$$Â = {D}^{\frac{1}{2}} ( A + l ) {D}^{\frac{1}{2}} \qquad ( 6 )$$

wherein H⁽¹⁾ is the node feature matrix of the l-th layer (each row corresponds to a node's feature vector), A is the normalized adjacency matrix A, D is the degree matrix, l is the identity matrix, W⁽¹⁾ is the l-th layer's learnable weight matrix, and σ is the activation function.
S22: Input preprocessed data into the GCN model and iteratively update node features via graph convolution to obtain final node representations encoding device information and interrelationships.
S23: Compute prediction errors by comparing model outputs to actual values using a predefined loss function (e.g., cross-entropy, mean squared error).
S24: Update GCN model weights via optimization algorithms (e.g., gradient descent, Adam) based on the loss gradient to minimize errors and enhance performance.
S25: Iteratively optimize the GCN model until a second iteration condition is met. Preferably, the second iteration condition is model convergence or reaching a preset iteration count.
S26: Output the trained GCN model.

It is noted that particular embodiments of the application are described above. Other embodiments are within the scope of the appended claims. In some cases, the actions or steps documented in the claims may be performed in a different order than in the embodiments and still achieve the desired results. In addition, the processes depicted in the accompanying drawings do not necessarily require the particular order or consecutive order shown to achieve the desired results. In some embodiments, multitasking and parallel processing is also possible or may be advantageous.

For ease of description, the above devices are described in terms of functionality into various modules described separately. Of course, it is possible to implement the functions of the various modules in the same or more software and/or hardware when implementing the present application.

It is further noted that the terms “including”, “comprising”, or any other variant thereof, are intended to cover non-exclusive inclusion such that a process, method, commodity, or apparatus comprising a set of elements includes not only those elements, but also other elements not expressly listed, such that the process, method, commodity, or apparatus includes not only those elements, but also other elements, includes other elements that are not expressly listed or that are inherent to such process, method, commodity or equipment. Without further limitation, the fact that an element is defined by the phrase “including a . . . ” does not preclude the existence of another identical element in the process, method, commodity or apparatus including said element.

The various embodiments of the present application are described in a progressive manner, whereby the same and similar parts of each embodiment are referred to each other, and each embodiment focuses on the differences from the other embodiments. In particular, for the system embodiment, since it is basically similar to the method embodiment, the description is relatively simple, and it is sufficient to refer to part of the description of the method embodiment where relevant.

It should be understood by those of ordinary skill in the art that the discussion of any of the above embodiments is merely exemplary and is not intended to imply that the scope of the present disclosure, including the claims, is limited to these examples; combinations between the above embodiments, or between technical features in different embodiments, are also possible within the contemplation of this disclosure, and the steps may be realized in any order and many combinations of different aspects of one or more embodiments of the present application, as described above, may exist, many other variations of different aspects of the multiple embodiments of the present application, which for the sake of brevity they are not provided in detail.

Although the present disclosure has been described in connection with specific embodiments of the present disclosure, many substitutions, modifications, and variations of these embodiments will be apparent to one of ordinary skill in the art in light of the preceding description.

One or more embodiments of the present application are intended to cover all such substitutions, modifications, and variations that fall within the broad scope of the appended claims. Accordingly, any omissions, modifications, equivalent substitutions, improvements, and the like made within the spirit and principles of the one or more embodiments of the present application shall be included within the scope of protection of the present disclosure.

1. An intelligent data platform for medical waste management and equipment monitoring, comprising:
a data collection module configured to collect medical waste information and equipment processing data;
a data processing module comprising a convolutional neural network module and a graph convolutional neural network module;
wherein the convolutional neural network module is configured to train and process the medical waste information, the medical waste information comprises one or more of solid category information, geographical location information, hospital information, department information, medical waste type information, department specific collection frequency information, and total quantities of various medical waste types;
wherein the graph convolutional neural network module is configured to train and process the equipment processing data, the equipment processing data comprising one or more of daily operation count of equipment, single-operation duration of equipment, pre-operation medical waste weight per equipment operation, preoperation medical waste volume per equipment operation, post-operation medical waste weight per equipment operation, and post-operation medical waste volume per equipment operation;
a data storage module configured to store trained and processed datasets;
a data feedback and monitoring module configured to provide feedback and monitoring based on the trained and processed datasets; and
a human-machine interaction terminal configured to display data and receive user input commands;
wherein steps for training and processing the medical waste information by the convolutional neural network module comprise:
performing data preprocessing on various types of information in the medical waste information;
constructing a convolutional neural network model, wherein the convolutional neural network model comprises a convolutional layer, a pooling layer, and a fully connected layer;
inputting preprocessed data into the convolutional neural network model, performing forward propagation calculations through the convolutional layer, the pooling layer, and the fully connected layer to obtain output data of the model;
calculating a difference between the output data of the model and actual values using a preset loss function;
updating weights and biases of the convolutional neural network model using an optimization algorithm based on a gradient of the loss function;
iteratively optimizing the convolutional neural network model until a first iteration condition is satisfied;
outputting the trained convolutional neural network model; wherein an output of the convolutional layer is:
$${O}_{i}^{( l )} = {\sum }_{j = 0}^{k - 1} {l}_{i + j}^{( l - 1 )} \cdot {K}_{j}^{( l )} \qquad ( 1 )$$
wherein Oᵢ^{(l)} is an output of the l-th convolutional layer at position i, Iᵢ₊ⱼ^{(l-1)} is an input of the (l−1)-th layer at position i+j, Kⱼ^{(l)} is a weight of the l-th layer's convolutional kernel at offset j, and k is a size of the convolutional kernel;
an output of an activation function is:
$${A}_{i}^{( l )} = f ( {O}_{i}^{( l )} ) \qquad ( 2 )$$
wherein f is the activation function, and Aᵢ^{(l)} is an output of the l-th layer at position i after passing through the activation function;
an output of the pooling layer is:
$${P}_{i}^{( l )} = {max}_{j = 0}^{s - 1} {A}_{i \times s + j}^{( l )} \qquad ( 3 )$$
wherein Pᵢ^{(l)} is an output of the l-th pooling layer at position i, s is a stride of the pooling operation, and Equation (3) represents selecting a maximum value within each region of size s;
an output of the fully connected layer is:
$$y = W \cdot x + b \qquad ( 4 )$$
wherein y is an output of the fully connected layer, W is a weight matrix, x is an input feature vector, and b is a bias term;
wherein steps for training and processing the equipment processing data by the graph convolutional neural network module comprise:
preprocessing each data item in the equipment processing data, wherein each equipment is represented as a node, and a feature vector of each equipment comprises respective data of the equipment;
constructing a graph convolutional neural network model;
inputting preprocessed data into the graph convolutional neural network model, propagating and updating node features layer-by-layer through graph convolutional operations until final node representations are obtained;
calculating a difference between output data of the model and actual values using a preset loss function;
updating weight parameters of the graph convolutional neural network model based on a gradient of the loss function;
iteratively optimizing the graph convolutional neural network model until a second iteration condition is satisfied; and
outputting the trained graph convolutional neural network model;
wherein formulas of the graph convolutional neural network model comprise:
$${H}^{( l + 1 )} = σ ( Â {H}^{( l )} {W}^{( l )} ) \qquad ( 5 )$$
$$Â = {D}^{\frac{1}{2}} ( A + I ) {D}^{\frac{1}{2}} \qquad ( 6 )$$
wherein H^{(l)} is a node feature matrix of the l-th layer, each row of which corresponds to a feature vector of a node, Â is a normalized version of an adjacency matrix A of the graph, D is a degree matrix, I is an identity matrix, W^{(l)} is a learnable weight matrix of the l-th layer, and σ is an activation function.

2. The intelligent data platform for medical waste management and equipment monitoring according to claim 1, wherein the first iteration condition is that the convolutional neural network model converges or reaches a preset number of iterations.

3. The intelligent data platform for medical waste management and equipment monitoring according to claim 1, wherein the second iteration condition is that the graph convolutional neural network model converges or reaches a preset number of iterations.
